(19) United States
(12) Patent Application Publication
(10) Pub. No.: US 2014/0350399 A1
(43) Pub. Date: Nov. 27, 2014

(54) METHOD FOR EXAMINING HUMAN OR ANIMAL TISSUE

(71) Applicant: Charite-Universitaetsmedizin Berlin, Berlin (DE)

(72) Inventors: Ingolf Sack, Berlin (DE); Jurgen Braun, Berlin (DE); Heiko Tzschatzsch, Berlin (DE)

(21) Appl. No.: 14/363,391

(22) PCT Filed: Dec. 10, 2012
(86) PCT No.: PCT/DE2012/200082

§ 371 Date: Jun. 6, 2014

(30) Foreign Application Priority Data
Dec. 21, 2011 (DE) 10 2011 089 401.2

(51) Int. Cl. A61B 8/08; A61B 8/00

(52) U.S. Cl. CPC A61B 8/485; A61B 8/4477; A61B 8/4209
USPC 600/438

(57) ABSTRACT

The invention relates inter alia to a method for examining human or animal tissue, wherein at least one time-harmonic mechanical excitation wave of predetermined excitation frequency is coupled into the tissue, the wave speed of a shear wave in the tissue caused by the mechanical excitation wave and having the frequency of the excitation wave is measured by means of an ultrasound method, and an elasticity measurement specifying the elastic properties of the tissue is determined using the measured wave speed.

The invention relates to a method for examining human or animal tissue, in particular living tissue.

In medical diagnostics, determining the viscoelastic properties is becoming ever more important within the scope of tissue examinations of living tissue. Thus, gathering elasticity values of e.g. the liver by means of elastography is currently the only possibility for a reliable and noninvasive classification of liver fibrosis [1]. In addition to application in the liver, the focus in the development of elastography lies in the field of characterizing breast tumors and in the fields of musculoskeletal and cardiac applications.

The invention is based on the object of specifying a method for examining human or animal tissue, by means of which the elastic properties can be established easily, but nevertheless accurately.

According to the invention, this object is achieved by a method having the features in accordance with patent claim 1. Advantageous embodiments of the method according to the invention are specified in dependent claims.

According to this, provision is made according to the invention for a method, in which at least one time-harmonic mechanical excitation wave is coupled into the tissue at a predetermined excitation frequency, the wave speed of a shear wave, caused by the mechanical excitation wave and having the frequency of the excitation wave, in the tissue is measured with the aid of an ultrasound method and an elasticity measurement value specifying the elastic properties of the tissue is determined using the measured wave speed.

A substantial advantage of the method according to the invention consists of the fact that, for example, it can be used for determining the elasticity in vivo of brain tissue for clinical-diagnostic applications. By way of example, this renders it possible to detect and provide early diagnosis of neurodegenerative processes which occur as accompanying symptoms of diverse neuronal diseases, such as multiple sclerosis, Alzheimer's and Parkinson's. Against the backdrop of an aging society, brain elastography, for example for early diagnosis of Alzheimer's and Parkinson's, is of high socioeconomic relevance.

In order, in particular in the case of measurements in the region of the cranium, to achieve ideal illumination of the cranial interior and a particularly high measurement accuracy, it is considered to be advantageous if a first ultrasound measuring method is performed for determining a first wave speed measured value, at least one second ultrasound measuring method is performed for determining at least one second wave speed measured value, wherein the at least two ultrasound measuring methods are performed from different positions and/or with different feed-in angles, to be precise simultaneously with at least two ultrasound probes or with time offset using one or more ultrasound probes, and the elasticity measurement value specifying the elastic properties of the tissue is determined using the first and the at least one further wave speed measured value.

In principle, the above-described measurement method, in particular the advantageous variant on the basis of at least two ultrasound measuring methods with different feed-in points and/or feed-in angles, can be used in any tissue, i.e. also in liver tissue for example.

As already explained above, it is considered to be particularly advantageous if the above-described measurement method is used for characterizing brain tissue. Accordingly, provision is made in accordance with a preferred embodiment of the method for the time-harmonic mechanical excitation wave to be coupled into brain tissue and for the ultrasound measuring method or methods to be performed on the brain tissue and for an elasticity measurement value specifying the elasticity of the brain tissue to be measured.

In order to obtain particularly precise measurement results, it is considered to be advantageous in brain tissue measurements if, in the case of at least one ultrasound measurement, an ultrasound probe is positioned in the region of one of the two lateral cranial bones or of one of the two temporal fenestrae of the cranium containing the brain tissue.

Moreover, in brain tissue measurements, it is considered to be advantageous if a longitudinal wave is produced as time-harmonic mechanical excitation wave, which longitudinal wave is coupled into the occiput (or on the rear side of the cranium) by means of a vibration unit. The vibration unit advantageously has a pivotable head rocker; for measurement purposes, the occiput is e.g. placed on the pivotable head rocker and the pivotable head rocker is pivoted in a periodic manner, as a result of which waves are produced and coupled into the brain tissue.

In a particularly preferred embodiment of the method, provision is made for a plurality of ultrasound measurements to be performed using a probe arrangement having a plurality of ultrasound probes, wherein at least one ultrasound probe is arranged in the region of one of the two lateral cranial bones, at least one probe is arranged in the region of the opposite lateral cranial bone and/or at least one probe is arranged in the region between the two lateral cranial bones.

It is also considered to be advantageous if two or more time-harmonic mechanical excitation waves are coupled into the tissue, wherein the excitation frequencies differ from one another, the wave speeds of the shear waves caused in each case by the mechanical excitation waves are measured, and at least one elasticity measurement value specifying the viscoelastic properties of the tissue is determined using the measured wave speeds and the frequency dispersion.

Particularly precise measurement results can be obtained on the basis of probability analysis; accordingly, it is considered to be advantageous if the wave speed of the shear waves or the wave speed of the shear waves are determined using probability analysis, in which probability density values are calculated for the preliminary wave speed measured values established during the measurement, and the final measurement value specifying the measured wave speed is calculated with the aid of the probability density values.

The invention moreover relates to an arrangement for examining human or animal tissue. According to the invention, the arrangement comprises: an excitation appliance, which is suitable for coupling at least one time-harmonic mechanical excitation wave with a predetermined excitation frequency into the tissue, at least one ultrasound probe and an evaluation appliance, which is connected to the ultrasound probe and suitable for measuring the wave speed of a shear wave caused by the mechanical excitation wave and having the frequency of the excitation wave, and determining an elasticity measurement value specifying the elastic properties of the tissue using the measured wave speed.

In respect of the advantages of the arrangement according to the invention, reference is made to the explanations above in the context of the method according to the invention, since the advantages of the method according to the invention substantially correspond to those of the arrangement according to the invention.

The excitation appliance preferably comprises a pivotable head rocker, which is suitable for coupling-in a time-harmonic mechanical excitation wave in the form of a longitudinal wave.

The probe arrangement preferably comprises a plurality of ultrasound probes, which allow ultrasound measuring methods to be performed from different positions and/or with different feed-in angles.

The probe arrangement particularly preferably has a bracket, on which the ultrasound probes are arranged in such a way that, after placing the bracket onto a cranium, at least one ultrasound probe is positioned in the region of one of the two lateral cranial bones, at least one probe is positioned in the region of the opposite lateral cranial bone and at least one probe is positioned in the region between the two lateral cranial bones.

The invention moreover relates to only a probe arrangement, comprising a bracket which is or can be fitted to a cranium, on which bracket a multiplicity of, at least three, ultrasound probes have been attached, of which probes at least one ultrasound probe is positioned in the region of one of the two lateral cranial bones, at least one probe is positioned in the region of the opposite lateral cranial bone and at least one probe is positioned in the region between the two lateral cranial bones if the bracket is placed onto the cranium as intended.

In respect of the advantages of the probe arrangement according to the invention, reference is made to the explanations above in the context of the method according to the invention, since the advantages of the method according to the invention substantially correspond to those of the probe arrangement according to the invention.

In the following text, the invention will be explained in more detail on the basis of exemplary embodiments; here, in an exemplary manner:

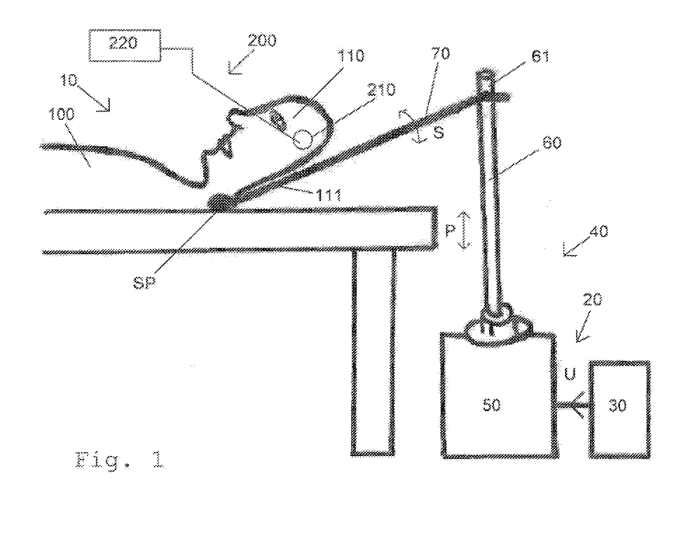

FIG. 1 shows an exemplary embodiment of an arrangement according to the invention for examining human or animal tissue,

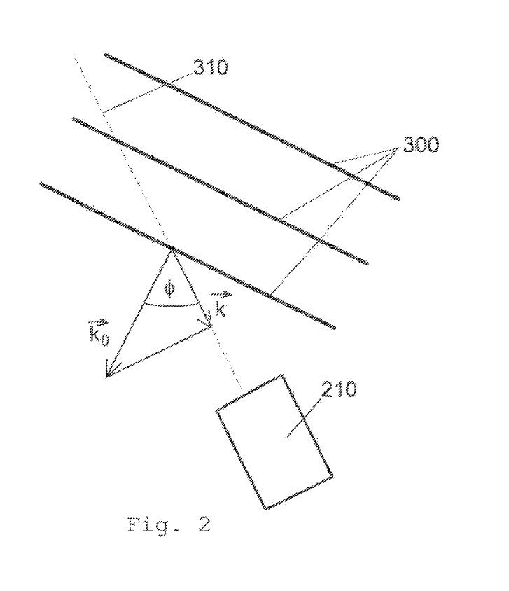

FIG. 2 shows shear wavefronts, the wave speed of which is measured with the aid of an ultrasound measuring method,

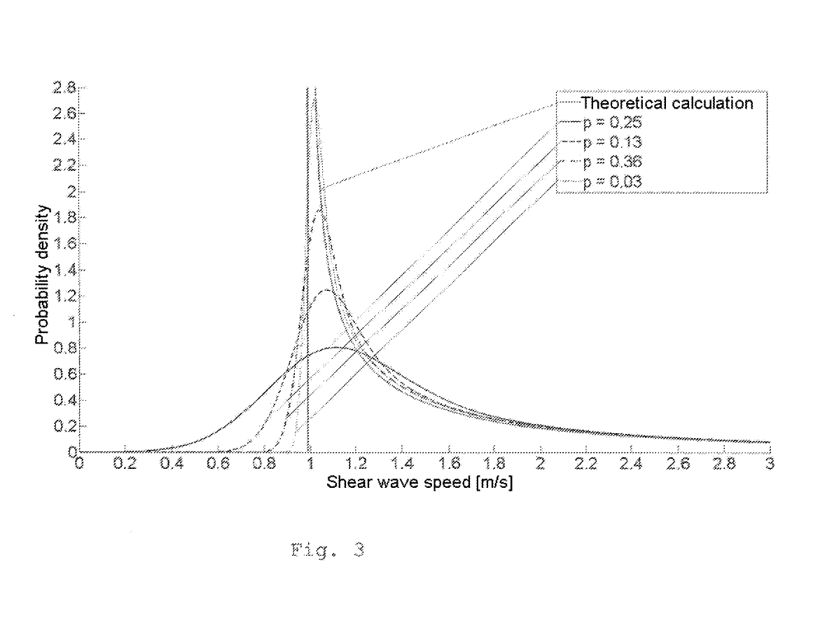

FIG. 3 shows histograms for simulated probability densities of shear wave speeds,

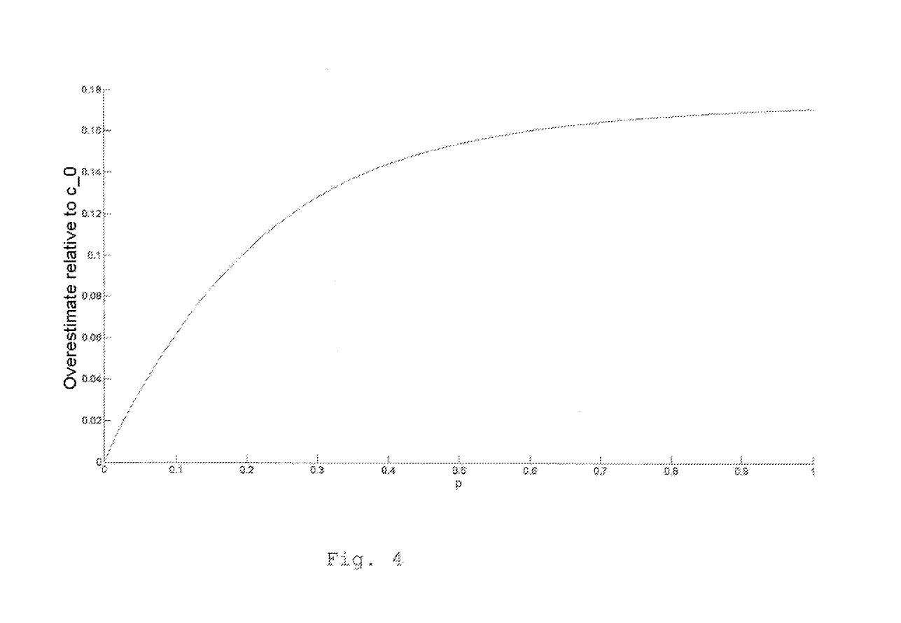

FIG. 4 shows a diagram depicting the relative overestimate of the shear wave speed depending on an error p,

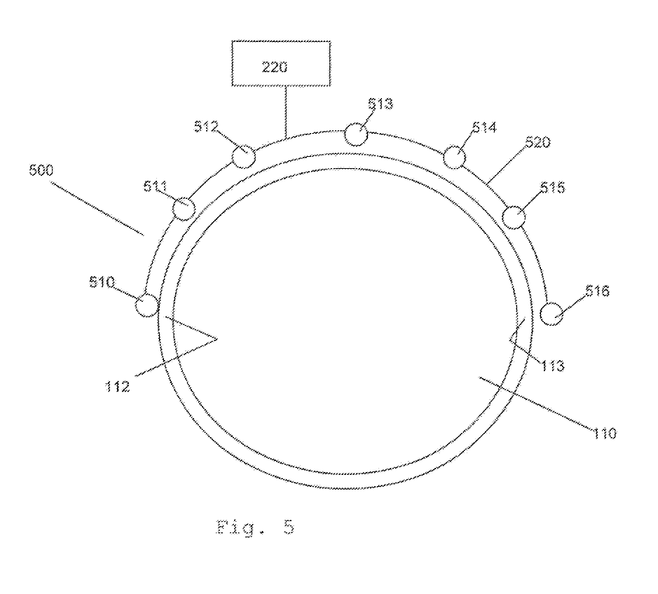

FIG. 5 shows an exemplary embodiment of a probe arrangement according to the invention, which can be used for examining human or animal tissue, and

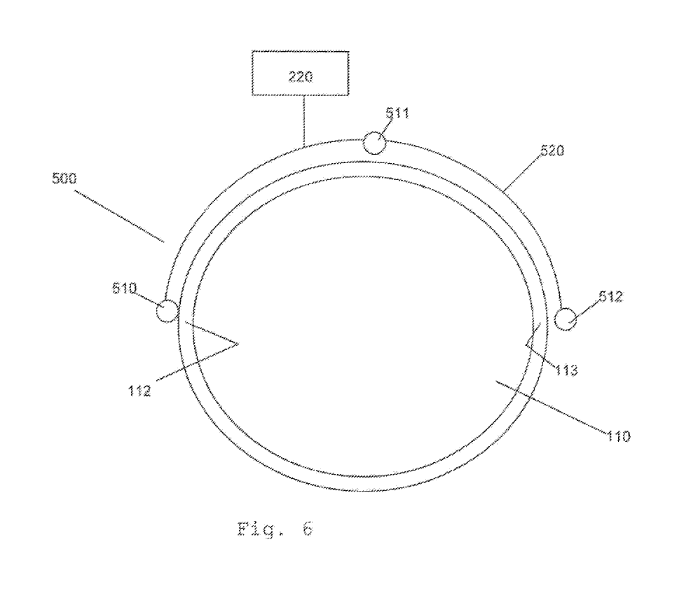

FIG. 6 shows a further exemplary embodiment of a probe arrangement according to the invention, which can be used for examining human or animal tissue.

In the figures, the same reference signs are, for reasons of clarity, always used for identical or comparable components.

FIG. 1 shows an exemplary embodiment of an arrangement 10 suitable for examining human or animal tissue. The arrangement 10 comprises an excitation appliance 20 comprising a frequency generator 30 and a vibration unit 40. The vibration unit 40 comprises a loudspeaker 50, a pole 60 and a pivotable head rocker 70.

The frequency generator 30 is connected to the loudspeaker 50, wherein the pole 60 is indirectly or directly coupled to the loudspeaker membrane of said loudspeaker. Mechanical vibrations of the loudspeaker membrane are transmitted to the pivotable head rocker 70 via the pole 60. To this end, one pole end 61 of the pole 60 is connected to the pivotable head rocker 70, which can be pivoted about a pivot point SP.

In FIG. 1, there can be seen a human 100, whose cranium 110 is placed on the pivotable head rocker 70. It can be seen that the rear side 111 of the cranium lies directly on the head rocker 70.

FIG. 1 moreover shows an ultrasound instrument 200, which comprises an ultrasound probe 210 and an evaluation appliance 220. In the illustration as per FIG. 1, the ultrasound probe 210 is positioned in the region of one of the two lateral cranial bones of the cranium 110. The ultrasound probe 210 is movable, and so the position thereof and the irradiation angle of the ultrasound beams emitted by the ultrasound probe 210 can be changed for successive ultrasound measurements. By way of example, the ultrasound probe 210 can produce time-resolved B-mode or M-mode ultrasound signals for the ultrasound measurements.

By way of example, the arrangement 10 in accordance with FIG. 1 can be operated as follows:

The frequency generator 30 produces one or, simultaneously, a plurality of AC voltages U, the frequency or frequencies of which preferably lies or lie in a frequency range between 10 and 100 Hz, particularly preferably between 25 and 80 Hz. The AC voltage U excites the loudspeaker membrane of the loudspeaker 50 to oscillate, as a result of which the pole 60 undergoes a vibration movement along the arrow direction P. The vibration movement of the pole 60 is converted into a pivoting movement of the pivotable head rocker 70, which begins to swing along the arrow direction S.

As a result of the head rocker 70 swinging, a time-harmonic mechanical excitation wave is coupled into the cranium 110 of the human 100. Since the rear side 111 of the cranium lies on the head rocker 70, a mechanical longitudinal wave, inter alia, is coupled into the cranium 110. The time-harmonic mechanical excitation wave coupled thus produces shear waves within the cranium 110 or within the brain tissue, which shear waves are depicted in an exemplary manner in FIG. 2.

In FIG. 2, the shear wavefronts of the shear waves are denoted by the reference sign 300. The shear waves form due to the time-harmonic mechanical excitation wave, which is not shown in FIG. 2 and which is coupled into the cranium 110 from the head rocker 70.

The wave speed of the shear wavefronts 300 depends on the elasticity of the brain tissue. Therefore, by measuring the wave speed of the shear wavefronts 300, it is possible to establish the elasticity of the brain tissue of the human 100 (cf. FIG. 1).

In order to measure the wave speed of the shear wavefronts 300, provision is made for the ultrasound probe 210 (cf. FIGS. 1 and 2), which couples an ultrasound beam 310 into the cranium 110 of the human 100 in the region of the lateral cranial bone. FIG. 2 schematically shows that there can be an angle between the propagation direction of the shear wavefront 300 and the ultrasound beam 310, which angle is denoted by the reference sign φ in FIG. 2. The propagation directions of the waves are denoted by the vectors k and k₀.

Establishing the wave speed of the shear wavefronts 300 and establishing the elastic properties of the brain tissue within the cranium 110 are now explained more closely in detail on the basis of an exemplary embodiment:

Due to the forced continuous mechanical excitation of the brain tissue by a harmonic vibration at the angular frequency ω₀, the introduced shear wave has a time-harmonic behavior:

u(x,t)=u₀(x)sin {ω₀t+θ(t)}. (1)

In the first signal processing step, the mechanical deflection u(x,t) can be reconstructed from the raw ultrasound signals I(x,t). In the M-mode, the time resolution of the signals I(x,t) corresponds to the pulse repetition rate Δt of the individual A-mode lines. Added to the real valued I(x,t) is the spatial Hilbert transform thereof as an imaginary part signal, from which the complex quadrature signal I*(x,t) emerges. Using I*(x,t) and the complex conjugate function Ī*(x,t) thereof, the complex correlation function in the depth window Δx is calculated,

$${R}^{*} ( Δ u ) = {\sum }_{X = x - \frac{Δ x}{2}}^{X = x + \frac{Δ x}{2}} {I}^{*} ( X + Δ u , t - Δ t ) {\overline{I}}^{*} ( X , t ) , \qquad ( 2 )$$

the phase angle of which can be minimized over the variation of the deflection difference Δu:

$$Δ u = {min}_{Δ u} \{ \vert ∠ {R}^{*} ( Δ u ) \vert \} . \qquad ( 3 )$$

From the deflection difference Δu, the deflection speed of the waves emerges as

$$\dot{u} ( x , t + \frac{Δ t}{2} ) = \frac{Δ u}{Δ t} , \qquad ( 4 )$$

from which, in turn, the sought-after deflection emerges for time-harmonic oscillations (see equation 1):

$$u ( x , t ) = \frac{\dot{u} ( x , t + π / 2 )}{{ω}_{0}} . \qquad ( 5 )$$

A Fourier transform can be used to calculate the complex signal at the excitation frequency u*(x,ω=ω₀), which is required to derive the shear wave speed. One option for determining the shear wave speed c is provided by the complex Helmholtz inversion. By means of the Helmholtz inversion, the complex shear modulus G*(x,ω₀) is initially calculated at the excitation frequency:

$${G}^{*} ( x , {ω}_{0} ) = - ρ {ω}_{0} \frac{{u}^{*} ( x , {ω}_{0} )}{\frac{{\partial }^{2} u}{\partial {x}^{2}} ( x , {ω}_{0} )} . \qquad ( 6 )$$

ρ represents the density of the examined material. The shear wave speed emerges as

$${c}^{*} = \sqrt{\frac{{G}^{*}}{ρ}} and \frac{1}{c} = Re ( \frac{1}{{c}^{*}} ) . \qquad ( 7 )$$

Each individual examination using a time-resolved ultrasound measurement method (e.g. M-mode) supplies at least one shear wave speed measured value c and at least one associated error value Δc. All measured values c are plotted in a histogram, which shows the probability density against the shear wave speed. An option for displaying the histogram of the measured values c consists in the superposition of normal Gaussian distribution curves at each measured value c with the full width at half maximum of the experimental error Δc.

FIG. 3 shows histograms for the simulated probability density of the shear wave speeds with an actual speed c₀=1 m/s. The analytical function was calculated using equation (10). FIG. 3 depicts simulated histograms with different error factors p and the analytical approximation for error-free measurements. In the simulations, the error factors p of the individual shear wave speeds were set to be proportional to the measured shear wave speed c:

Δc=pc with p>0. (8)

Due to the geometric projection of the wavenumber k₀ of the shear wave on the direction vector of the ultrasound beam, k≧k₀ applies for the measured wavenumber, i.e. the apparent wave speed of an individual measurement (c) is overestimated by

$$c = \frac{{c}_{0}}{cos φ} , \qquad ( 9 )$$

where φ specifies the gating angle (cf. FIG. 2).

Due to the gating angle φ, the maximum appears at the wave speed cₘₐₓ≧c₀. The case c=c₀ only applies for φ=0, i.e. where the ultrasound beam impinges perpendicularly on the wavefront (see FIG. 2). For error-free measurements (Δc=0), the probability density ρ_{W}(c) can be derived as follows:

$${ρ}_{W} ( c ) = \frac{2}{π c \sqrt{{( \frac{c}{{c}_{0}} )}^{2} - 1}} . \qquad ( 10 )$$

By means of numerical simulations, it is possible to estimate the relative overestimate of the shear wave speed in the histogram. FIG. 4 shows the relative overestimate of the shear wave speed depending on the error factor p.

The proposed probability analysis of the wave speeds requires a certain minimum number of individual measurements. By assuming planar shear waves and a uniform angle distribution φ over the 180° sector of all possible probe positions, it is possible to estimate said minimum number. According to (9), the following emerges for the relative deviation from the actual shear wave speed:

$$\frac{c - {c}_{0}}{c} = 1 - cos φ . \qquad ( 11 )$$

If a maximum relative deviation of 5% is now permitted, the minimum angle increment Δφ=18° emerges from (11) from 1-0.05>cos Δφ. This corresponds to a minimum number of 10 individual measurements for the proposed probability analysis of the wave speeds.

After determining the actual wave speed c, it is then possible to determine the elasticity, in particular the viscoelasticity of the brain tissue, to be precise e.g. with the aid of one or more of the following model functions for the frequency-dependent complex modulus G*, by means of which the frequency-dependence of the established wave speeds can be adapted:

$$c ( ω ) = \frac{1}{Re [ \sqrt{\frac{ρ}{{G}^{*} ( ω )}} ]} . \qquad ( cf . equation 7 )$$

By way of example, G^{*} can be derived using the following viscoelastic models:

$${G}^{*} ( ω ) = \{ \begin{matrix}μ + i ω η & Voigt \\ \frac{i ω η μ}{μ + i ω η} & Maxwell \\ \frac{{μ}_{1} {μ}_{2} + i ω η ( {μ}_{1} + {μ}_{2} )}{ {μ}_{2} + i ω η} & Zener \\ - ω {η}_{1} \frac{ω {η}_{2} - i μ}{μ + i ω ( {η}_{1} + {η}_{2} )} & Jeffreys \\ {{μ}^{1 - α} ( i ω η )}^{α} & springpot\end{matrix}$$

Here, μ and η respectively represent the shear modulus and shear viscosity characteristics.

FIG. 5 shows an exemplary embodiment of a probe arrangement 500 according to the invention, which can be used for measuring the elastic properties of the brain tissue situated in the cranium 110 (cf. FIG. 1) instead of the individual probe 210 and which is particularly suitable for this.

The probe arrangement 500 comprises seven ultrasound probes, which are denoted by reference signs 510 to 516. The seven ultrasound probes 510 to 516 are assembled on a bracket 520 of the probe arrangement. The bracket 520 is preferably formed in such a way that it can be placed onto the cranium 110 of the human in such a way that at least two of the ultrasound probes are situated in the region of the two lateral cranial bones 112 and 113 or in the region of the temporal fenestrae of the cranium 110. Thus, it can be seen in FIG. 5 that the ultrasound probe 510 in FIG. 5 is arranged in the region of the left-hand lateral cranial bone 112 of the cranium 110 and the ultrasound probe 516 in FIG. 5 is arranged in the region of the right-hand lateral cranial bone 113 of the cranium 110 when the bracket 520 or the probe arrangement 500 overall have been placed onto the cranium 110 as intended.

The probe arrangement 500 renders it possible to simultaneously couple ultrasound beams into the cranium 110 at different positions of the cranium 110, wherein the irradiation direction of the ultrasound beams differs. Thus, it can be seen that the beam direction of the ultrasound probe 510 extends substantially in the direction of the right-hand lateral cranial bone 113 from the left-hand lateral cranial bone 112, whereas the alignment of the ultrasound beam of the ultrasound probe 513 is substantially perpendicular thereto.

Therefore, it is very easily and quickly possible to simultaneously carry out a plurality of ultrasound measurement methods using the probe arrangement 500, which can be placed onto the cranium 110 by means of the bracket 520, wherein the location of coupling-in the ultrasound beams and the feed-in angle of the ultrasound beams are different. In this manner, it is possible to produce a plurality of wave speed values within a very short time, which wave speed values—as explained above in detail in conjunction with FIGS. 2 to 4—are preferably evaluated on the basis of probability analysis.

FIG. 6 shows a further exemplary embodiment of a probe arrangement 500 according to the invention, which can be used for measuring the elastic properties of the brain tissue situated in the cranium 110 (cf. FIG. 1) instead of the ultrasound probe 210. The probe arrangement 500 comprises three ultrasound probes, which are denoted by reference signs 510 to 512. The three ultrasound probes 510 to 512 are assembled on a bracket 520 of the probe arrangement. The bracket 520 is formed in such a way that it can be placed onto the cranium 110 of the human in such a way that at least two of the ultrasound probes are situated in the region of the two lateral cranial bones 112 and 113 or in the region of the two temporal fenestrae of the cranium 110 when the bracket 520 or the probe arrangement 500 overall have been placed onto the cranium 110 as intended. The central ultrasound probe 511 is situated—preferably centrally—between the two outer ultrasound probes 510 and 512.

### LIST OF REFERENCE SIGNS

10 Arrangement
20 Excitation appliance
30 Frequency generator
40 Vibration unit
50 Loudspeaker
60 Pole
61 End of the pole
70 Head rocker
100 Human
110 Cranium
111 Rear side of the cranium
112 Cranial bone
113 Cranial bone
200 Ultrasound instrument
210 Ultrasound probe
220 Evaluation appliance
300 Shear wavefront
310 Ultrasound beam
500 Probe arrangement
510-516 Ultrasound probes
520 Bracket
35
k Measured wavenumber vector
k₀ Actual wavenumber vector
p Error factor
P Arrow direction
S Arrow direction
SP Pivot point
U AC voltage
φ Angle

1. A method for examining human or animal tissue, wherein
at least one time-harmonic mechanical excitation wave is coupled into the tissue at a predetermined excitation frequency,
the wave speed of a shear wave, caused by the mechanical excitation wave and having the frequency of the excitation wave, in the tissue is measured with the aid of an ultrasound method and
an elasticity measurement value specifying the elastic properties of the tissue is determined using the measured wave speed.

2. The method as claimed in claim 1,
characterized in that
a first ultrasound measuring method is performed for determining a first wave speed measured value,
at least one second ultrasound measuring method is performed for determining at least one second wave speed measured value,
wherein the at least two ultrasound measuring methods are performed from different positions and/or with different feed-in angles, to be precise simultaneously with at least two ultrasound probes or with time offset using one or more ultrasound probes, and
the elasticity measurement value specifying the elastic properties of the tissue is determined using the first and the at least one second wave speed measured value.

3. The method as claimed in claim 1,
characterized in that
the time-harmonic mechanical excitation wave is coupled into brain tissue and the ultrasound measuring method or methods is/are performed on the brain tissue and
an elasticity measurement value specifying the elasticity of the brain tissue is measured.

4. The method as claimed in claim 1,
characterized in that
an ultrasound probe (210) is positioned in the region of one of the two lateral cranial bones (112, 113) of the cranium (110) containing the brain tissue.

5. The method as claimed in claim 1,
characterized in that
a longitudinal wave is produced as time-harmonic mechanical excitation wave, which longitudinal wave is coupled into the occiput or the rear side (111) of the cranium by means of a vibration unit (40), in particular by means of a pivotable head rocker (70).

6. The method as claimed in claim 1,
characterized in that
a plurality of ultrasound measurements are performed using a probe arrangement (500) having a plurality of ultrasound probes,
wherein at least one ultrasound probe (510) is arranged in the region of one of the two lateral cranial bones (112), at least one probe (516, 512) is arranged in the region of the opposite lateral cranial bone (113) and/or at least one probe (511-515, 511) is arranged in the region between the two lateral cranial bones (113).

7. The method as claimed in claim 1,
characterized in that
two or more time-harmonic mechanical excitation waves are coupled into the tissue, wherein the excitation frequencies differ from one another,
the wave speeds of the shear waves caused in each case by the mechanical excitation waves are measured, and
at least one elasticity measurement value specifying the viscoelastic properties of the tissue is determined using the measured wave speeds.

8. The method as claimed in claim 1,
characterized in that
the wave speed of the shear wave or the wave speed of the shear waves are determined using probability analysis, in which probability density values are calculated for the preliminary wave speed measured values established during the measurement, and
the final measurement value specifying the measured wave speed is calculated with the aid of the probability density values.

9. An arrangement (10) for examining human or animal tissue, comprising
an excitation appliance (20), which is suitable for coupling at least one time-harmonic mechanical excitation wave with a predetermined excitation frequency into the tissue,
at least one ultrasound probe (210, 510-516) and
an evaluation appliance (220), which is connected to the ultrasound probe and suitable for measuring the wave speed of a shear wave caused by the mechanical excitation wave and having the frequency of the excitation wave, and determining an elasticity measurement value specifying the elastic properties of the tissue using the measured wave speed.

10. The arrangement as claimed in claim 9,
characterized in that
the excitation appliance (20) comprises a pivotable head rocker (70), which is suitable for coupling-in a time-harmonic mechanical excitation wave in the form of a longitudinal wave, and
a probe arrangement (500) has a plurality of ultrasound probes (510-516), which allow ultrasound measuring methods to be performed from different positions and/or with different feed-in angles,
wherein the probe arrangement has a bracket (520), on which the ultrasound probes are arranged in such a way that, after placing the bracket onto a cranium (110), at least one ultrasound probe is positioned in the region of one of the two lateral cranial bones (112), at least one probe is positioned in the region of the opposite lateral cranial bone (113) and at least one probe is positioned in the region between the two lateral cranial bones.

11. A probe arrangement (500), comprising a bracket (520) which is or can be fitted to a cranium (110), on which bracket a multiplicity of, at least three, ultrasound probes (510-516) have been attached, of which probes at least one ultrasound probe is positioned in the region of one of the two lateral cranial bones (112), at least one probe is positioned in the region of the opposite lateral cranial bone (113) and at least one probe is positioned in the region between the two lateral cranial bones if the bracket is placed onto the cranium as intended.
